(19) United States
(12) Patent Application Publication
(10) Pub. No.: US 2023/0392122 A1
(43) Pub. Date: Dec. 7, 2023

(54) LONG-TERM AND FUNCTIONAL CULTURE OF HEPATIC ORGANOIDS (eHEPO) DERIVED FROM EPCAM+ ENDODERMAL PROGENITOR CELLS DIFFERENTIATED FROM INDUCED PLURIPOTENT STEM CELLS

(71) Applicants: DOKUZ EYLUL UNIVERSITESI REKTORLUGU, Izmir (TR); IZMIR BIYOTIP VE GENOM MERKEZI, Izmir (TR); KOC UNIVERSITESI, Istanbul (TR)

(72) Inventors: Serife Esra ERDAL BAGRIYANIK, Izmir (TR); Soheil AKBARI, Izmir (TR); Tevfik Tamer ONDER, Istanbul (TR); Nur ARSLAN, Izmir (TR)

(73) Assignees: DOKUZ EYLUL UNIVERSITESI REKTORLUGU, Izmir (TR); IZMIR BIYOTIP VE GENOM MERKEZI, Izmir (TR); KOC UNIVERSITESI, Istanbul (TR)

(21) Appl. No.: 18/247,651

(22) PCT Filed: Aug. 2, 2021
(86) PCT No.: PCT/TR2021/050761

§ 371 Date: Apr. 3, 2023

(30) Foreign Application Priority Data
Oct. 8, 2020 (TR) 2020/16056

(51) Int. Cl. C12N 5/071

(52) U.S. Cl. CPC C12N 5/0671; C12N 2506/45; C12N 2513/00; C12N 2500/02; C12N 2500/40; C12N 2500/38; C12N 2501/345; C12N 2501/11; C12N 2501/385; C12N 2501/115; C12N 2501/12; C12N 2501/155; C12N 2501/39; C12N 2501/415; C12N 2501/42

(57) ABSTRACT

A three-dimensional (3D) liver organoid obtained through the differentiation of induced pluripotent stem cells (IPSCs) in laboratory culture medium and a production method of the 3D hepatic organoid are provided. The production method includes the following steps: differentiating the IPSCs into a definitive endoderm in a medium containing Activin A, Wnt3a, and R-spo1 factors; adding 5 ng/ml R-spo 1 during IPSC differentiation to increase an amount of EpCAM+endoderm progenitor cells; cell sorting of the EpCAM+endoderm progenitor cells through a fluorescence-activated cell sorting (FACS) method; culturing a sorted EpCAM+endoderm progenitor cells in a 3D Matrigel medium of 37° C., 5% CO2, 95% humidity and 7.2-7.5 pH.

### CROSS REFERENCE TO THE RELATED APPLICATIONS

This application is the national phase entry of International Application No. PCT/TR2021/050761, filed on Aug. 2, 2021, which is based upon and claims priority to Turkish Patent Application No. 2020/16056 (TR), filed on Oct. 8, 2020, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBUY160_Sequence_Listing.txt, created on 03/23/2023, and is 1,697 bytes in size.

### TECHNICAL FIELD

The invention relates to three-dimensional liver organoids obtained through the differentiation of induced pluripotent stem cells (IPSCs) in laboratory culture medium.

### BACKGROUND

Organoids are three-dimensional small structures which are structurally and functionally organ-like, and they are generated under laboratory conditions from organ-specific adult stem cells or pluripotent stem cells.

Organoids form by mimicking the self-organisation processes seen during embryo development. These main processes are: the specific separation of cells (cell sorting out) and the gravitation of the cell towards its future cell type, depending on its spatial location (lineage commitment).

Patent document No US 2018/0258400 A1 relates to the formation of three-dimensional liver organoids from pluripotent stem cells. Liver organoids are obtained from multipotent endoderm spheroid progenitor cells derived from pluripotent stem cells. Endoderm cells are obtained in 8 days; 3D hepatic organoid lineage stage takes 47 days and functional hepatic organoid culture lasts 68 days. The designated organoid culture medium requires more growth factor, which consequently increases the cost. In addition to the growth factors for the organoid stage, there is also a need for the commercially available HCM (LONZA) medium. Not only is this an expensive medium, but the exact amount of its components is not known since it is a commercial product, and it also does not have an alternative. In order to form an organoid, you need to start with a single cell, form 3D spheroid structures, then convert it back to a single cell again to form an organoid. These procedures require a lot of time, effort and expenditure. Furthermore, the functionality and cellular content of the organoids in a late passage is not known, and it is stated that organoids can be cultured for a maximum of 4.5 months. It is necessary to set off with a heterogeneous population to form the organoid culture.

Patent document No US 2019/0298775 A1 relates to the formation of liver organoids from pluripotent stem cells. In addition to the growth factors for the organoid stage, there is also a need for the commercially available HCM (LONZA) medium. Not only is this an expensive medium, but the exact amount of its components is not known as it is a commercial product. There is no data on the functionality and cellular content of the organoids in a late passage. It is necessary to set off with a heterogeneous population to form the organoid culture. Organoid formation last 28 days and can be cultured for a maximum of 30 days more.

Various growth factors and chemicals are used in iPSC-based organoid formation protocols. As not all cells are in the same state during this process, they differentiate in different ways. Some cells differentiate one way at a later stage, whereas others do not differentiate or do so in a less extent. This situation creates a heterogeneous population. Therefore, the result is setting off with cells of different potential during organoid formation, which consequently creates the possible existence of different cell types or progenitors derived from the endoderm in the obtained organoid structure.

For these reasons, there is a need for organoids which are obtainable more quickly, highly efficient, able to be cultured for a long time, able to carry out liver functions for longer periods, cost-effective and not commercially dependent.

### BRIEF SUMMARY OF THE INVENTION

The invention relates to the creation of three-dimensional cell/tissue culture which is known as organoid technology. It involves generating the functional liver (hepatic) organoid culture from EpCAM+ endodermal progenitor cells obtained through the differentiation of induced pluripotent stem cells under laboratory conditions. This culture has superiority over other hepatic organoids obtained from induced pluripotent stem cells (IPSCs) for the following reasons: a) it forms in just 14 days, b) it can be increased in culture in a proper way for over a year, c) it is able to carry out specific liver functions such as albumin production, glycogen storage, cellular ingestion of low-density lipoprotein (LDL) and cytochrome p450 enzyme activity, even in late passages. For this reason, this technology referred to as eHEPO will be rather convenient in terms of personalised drug screening, preclinical hepatotoxicity analyses and disease modelling.

The start to the invention was given by using a homogenous cell group which would allow the isolation of EpCAM positive cells at the endoderm differentiation stage and enable the formation of liver-specific cells only. This shows the source of the organoids and what kind of a population they are.

The invention demonstrates that the organoid formation percentage using EpCAM+ cells isolated from the endoderm is a minimum of 35%, and that EpCAM− cells do not form organoids. The organoid acquisition duration using the present technique is rather long due to the fact that organoids are obtained from all endoderm cells, and the culture cannot be run for a long period of time.

It is possible to obtain hepatic organoids in IPSCs in 14 days. An extra 10 days of differentiation is required for the formation of functional liver hepatocyte-like cells. In other words, hepatocytes with liver-specific functionality such as albumin secretion, cellular ingestion of LDL, fat storage, glycogen storage and drug detoxification enzyme activity are obtained at the end of the 25^{th} day.

In addition, the eHEPO technology was used in the modelling of citrullinemia, a rare metabolic liver disease. Through culturing, it was possible to mimic the exact phenotype seen in humans (like the liver not being able to eliminate ammonia).

The objective of the invention is to generate liver organoids from pluripotent stem cells. This invention enables the formation of liver organoids with the help of skin biopsies taken from healthy volunteers, as well as those with genetic liver diseases. The benefit of its technique is that the organoids are obtainable in a short time, that they still remain normal in long-term cultures and that they have liver functionality as disease phenotypes. Organoids obtained with this technology are advantageous in that they can be used in hepatotoxicity and drugs screenings.

Through this invention, the endoderm can be obtained in 5 days, the 3D hepatic organoid lineage in 10 days and the functional hepatic organoid in 25 days.

The culture of organoids is possible in an endoderm-derived hepatic organoid (eHEPO) culture up to the AT passage 48 for approximately 16-18 months, without losing their functionalities.

The invention involves sorting of the EpCAM cells and starting with a more pure and potential cell population, thus obtaining a more productive culture.

Through the method of the invention, an attempt was made to form organoids with EpCAM+ and EpCAM− cells. The former enabled 35-40% organoid formation, whereas the latter did not form any organoids.

Within the scope of the invention, the characterisation and functionality of organoids were tested from early passage phases to later stages.

With the invention, it is possible to obtain organoids which are not dependent on commercial products like HCM, and which preserve their cellular content and functionality after long-duration cultures.

During organoid formation, EpCAM, the more pure and effective cell, was isolated and, thus, the organoid was formed. This enables a quicker and more effective way of organoid formation.

In addition, this method was used in the modelling of citrullinemia, which is a urea cycle disorder. In terms of the organoids formed from the IPSCs obtained from dermal fibroblasts taken from two citrullinemia patients, it was observed that ammonia elimination mimicking the disease phenotype was not possible.

The invention creates an easy and robust protocol to model all liver metabolic diseases in vitro.

Through this invention, three-dimensional hepatic (liver) organoids were generated to be used in research towards the gene therapy planning and the discovery of drugs for the treatment of all liver metabolic diseases.

The invention specifically was used to mimic Citrullinemia phenotype in vitro and this was recently the most reliable human model for Citrullinemia as a drug screening platform.

The invention can be utilized as a platform for drug hepatotoxicity assay of the drug candidates.

The invention provides a live hepatic function monitoring system to follow any harmful effect on human liver

### BRIEF DESCRIPTION OF THE DRAWINGS

FIGS. 1A-1C: Organoid formation from IPSC-based EpCAM (+) progenitor cells. FIG. 1A) The protocol of forming organoid culture is summarised in this diagram. FIG. 1B) Immunofluorescence images of the expression of OCT3/4 (pluripotent), FOXA2, SOX17 and EpCAM (endoderm) markers at different stages (20× magnification) (upper panel). The expression of EpCAM and CXCR4 proteins on the 5^{th} day using the flow cytometry method. Endoderm differentiation productivity was tested in terms of three different healthy IPSCs (WT1, WT2, WT3) EpCAM and CXCR4 (bottom panel). FIG. 1C) Flow cytometry analyses show that R-spo1 causes an increase in the number of EpCAM (+) cells. The assays were repeated with three different healthy IPSCs (upper panel). The effect of R-spo1′ in IPSC-based endoderm cell morphology. DIC images; Differential interference contrast (bottom panel).

FIGS. 2A-2E: The formation of eHEPO organoids. FIG. 2A) The organoid-forming potential of EpCAM+ and EpCAM− cells. Images of the developed organoids at various time points using the phase contrast microscopy. “p” indicates the passage number. FIG. 2B) The phenotypic characterisation of endoderm-based healthy organoids at different passages (p6, p21 and p48) in an expansion medium (EM). FIG. 2C) Confocal images of EpCAM, HNF4a and ZO1 proteins. Cell nuclei were stained with DAPI. In p10 organoids, the whole EpCAM\HNF4α and ZO-1 structure was stained. Other staining was done in frozen sections. FIG. 2D) In the organoids, AFP and CK18 were stained with the immunohistochemistry method. H&E indicates haematoxylin/eosin staining. FIG. 2E) It shows the change in gene expression during the GSEA plot differentiation stage. Normalised enrichment scores (NES) and FDR q-values were analysed for each gene list.

FIGS. 3A-3G: In vitro differentiation of eHEPO organoids into mature hepatocytes. FIG. 3A) Confocal images of CK18, E-Cadherin, A1AT, ZO1 and ALB proteins in organoids cultured in differentiation medium (DM). Cell nuclei were stained with DAPI. In the p10 dataset, whole-mount staining was conducted on CK18, E-CAD/A1AT and ZO-1/ALB in the organoids, other conditions were stained in frozen sections. FIG. 3B) Immunohistochemistry images of the ALB, CK19 and E-cadherin proteins in the organoids. FIG. 3C) Images of the IPSCs taken with scanning electron microscopy. The arrow indicates apoptotic and multivesicular bodies (upper panel). The white circle and arrow indicate cellular connections and apical villuses (bottom panel). FIG. 3D) Lentiviral albumin promoter-GFP reporter system was applied to observe the albumin expression in the organoids. Images of the differentiation stages of IPSCs carrying pALB-GFP reporter using light microscopy and fluorescence microscopy. Flow cytometry shows the numbers of ALB+ cells within the organoid. FIG. 3E) GSEA plot demonstrating the different expressions of the genes between EM and DM. Normalised enrichment scores (NES) and FDR q-values were analysed for liver-specific genes. FIG. 3F) The heat map shows the expression of EM and DM related genes. FIG. 3G) qPCR analysis of the genes in EM, DM organoids and liver tissue. Fold changes were made between EM/DM and tissue/EM (4 technical replicates made for 3 different biologic replicates). (*p≤0.05).

FIGS. 4A-4E: In vitro functionality tests of eHEPO organoids. FIG. 4A) ELISA analysis of the albumin secretion of healthy organoids in EM and DM conditions at different passages (p6, p23, p48). Data was calculated upon taking an average of three different assays (ngALB/day/million cells). FIG. 4B) The CYP3A4 activity of the organoids was expressed as RLU/ml/million cells. FIG. 4C) Immunofluorescence images of LDL uptake on the 14^{th} day of differentiation at p10 and p48 passages. FIG. 4D) PAS staining images of glycogen storage on the 14^{th} day of differentiation at p10 and p48 passages. EM was used as negative control. FIG. 4E) Immunohistochemical staining images of sections obtained from organoid-transplanted livers after damaging NGS mice liver with DMN. The presence of GFP+ and ALB+ cells demonstrates that hepatocytes are engrafted in mice liver. (*p≤0.05; **p≤0.01; ***p≤0.001).

FIGS. 5A-5H: The formation and characterisation of IPSCs from citrullinemia (CTLN) patients. FIG. 5A) Morphological images of healthy, CTLN1 and CTLN2 IPSCs. FIG. 5B) Sequence analysis of exon 15 mutations in the ASS1 gene in fibroblasts and IPSCs obtained from healthy individuals as well as patients. FIG. 5C) PCR integration analyses of the episomal reprogramming vectors. FIG. 5D) Karyotype analyses of healthy, CTLN1 and CTLN2 IPSCs. FIG. 5E) NANOG, OCT4 and SSEA immunofluorescence images of IPSCs. Cell nuclei were stained with Hoechst (Scale: 100 μm). FIG. 5F) mRNA level expression analyses of pluripotent genes. FIG. 5G) Western blot analyses of the overexpression of GFP and ASS1 in healthy and CTLN1 IPSCs. FIG. 5H) Teratoma analyses of CTLN1 and CTLN2 IPSCs in SCID mice (Scale, 100 μm).

FIGS. 6A-6F: Citrullinemia disease modelling with eHEPOs. FIG. 6A) Light microscopy images of CTLN organoids. FIG. 6B) HNF4α, CK18, ZO-1, CK19 and ALB immunofluorescence staining in CTLN organoids. Cell nuclei were stained with DAPI. FIG. 6C) In vitro functionality tests of CTLN-GFP and CTLN-ASS-O/E organoids. ELISA analysis of the albumin secretion of CTLN1-GFP and CTLN1-ASS-O/E organoids at p10. FIG. 6D) PAS and LDL analyses of CTLN1-GFP and CTLN1-ASS-O/E organoids at p10. FIG. 6E) Heat map of healthy and CTLN IPSCs, endoderms and organoids. FIG. 6F) Ammonia detoxification capacity measurement of GFP (control) or ASS1-O/E which are overexpressed in healthy and disease-based mature eHEPOs. The assays were repeated three times and presented as μg/day/million cells (*p≤0.05; **p≤0.01; ***p≤0.001).

FIG. 7: Results of the urea level measurements in the medium.

### DETAILED DESCRIPTION OF THE INVENTION

The invention comprises the operational steps of the production method of three-dimensional hepatic organoids obtained from induced pluripotent stem cells (IPSCs):
The differentiation of IPSCs into a definitive endoderm in a medium containing Activin A, Wnt3a, and R-spo1 factors,
Adding 5 ng/ml R-spo 1 during IPSC differentiation to increase the amount of EpCAM+ endoderm cells,
Cell sorting of the EpCAM+endodermal progenitor cells through the fluorescence-activated cell sorting (FACS) method,
Culturing the sorted EpCAM+endodermal progenitor cells in a 3D Matrigel medium of 37° C., 5% CO2, 95% humidity and 7.2-7.5 pH,
Once the Matrigel solidifies, cell culture medium of 1.25 mM N-acetylcysteine with 1% N2 and 1% B27 without retinoic acid, 10 nM gastrin and 50 ng/ml EGF, 10% RSPO1 is added, 100 ng/ml FGF 10, 25 ng/ml HGF, 10 mM Nicotinamide, 5 uM A8301, DMEM/F12 with added 10 uM FSK culture medium is added; during the first 3 days of this step, 25 ng/ml Noggin and 30% Wnt CM and 10 uM Y27632 is added to the culture medium,
For the functional hepatocyte differentiation of the organoids, the medium needs to be changed with 1% N2 and 1% B27 without retinoic acid, 10 nM gastrin and 50 ng/ml EGF, 100 ng/ml FGF 10, 25 ng/ml HGF, 500 nM A8301, 10 uM DAPT, 25 ng/ml BMP7 and 30 uM Dexamethasone added developed cell culture medium DMEM/F12 containing differentiation medium.

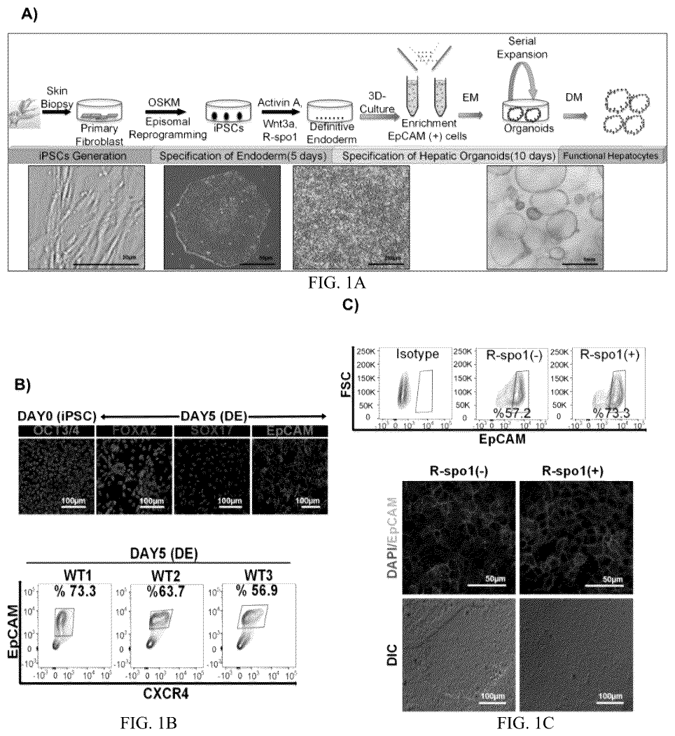

To start with, IPSCs were differentiated into definitive endoderms in a medium containing 100 ng/mL Activin A, 50 ng/mL Wnt3a, and 5 ng/mL R-spo1factors for 5 days (FIG. 1A). During the definitive endoderm development stage, the parenchymal cells of the liver derive from a specific germ layer. In addition to the morphological change, immunofluorescence staining shows that while IPSCs express pluripotent marker OCT3/4 before differentiation, the expression of this gene decreases once the endoderm forms at the end of day 5, and that only the expression of certain genes like SOX17, FOXA2 and EpCAM increase. In addition, flow cytometry analyses revealed that 65% of the population was CXCR4+/EpCAM+. In particular, it is evident that following independent differentiations, the percentage of CXCR4+/EpCAM+ endodermal cells derived from three independent IPSC lines (WT1, WT2 and WT3) is not too different from one another, and that the endodermal induction step of the protocol can be repeated (FIG. 1B). At this point of the assay, the differentiation medium was modified to obtain more EpCAM+ endoderm cells. Wnt signalling pathway agonist and 5 ng/ml R-spo1, which is known to play a significant role in stem cell growth, were added to the endoderm medium. While this modification increases the EpCAM ratio considerably, the morphology of the cells is not affected negatively (FIG. 1C). As a result, this protocol enables IPSCs to differentiate into more EpCAM+endoderm cells in two-dimensional mediums.

Afterwards, EpCAM+ cells obtained as a result of the two-dimensional differentiation of IPSCs were sorted through fluorescence-activated cell sorting (FACS) in medium culturing organoids from adult liver, and the EpCAM+ or EpCAM− endodermal progenitor cells were cultured in 3D Matrigel medium of 37° C., 5% CO2, 95% humidity and 7.2-7.5 pH. IPSC-derived endoderm cell suspensions were counted with Trypan blue to determine the number of live cells, then the cells were thawed 1×106 cell/1 ml with a sorting buffer (1×PBS, 1 mM EDTA, 25 mM HEPES pH 7.0, 1% FBS, 0.2 μm filtered and kept at +4° C.) and strained through a 100 um mesh. Following being centrifuged, the pellet was thawed with 100 ul buffer for 107 cells, and incubated with anti-CD326 (EpCAM)-FITC with a ratio of 1:11 for 10 minutes at 4° C. After the washes, the pellet was thawed 1×106 cell/1 ml with a sorting buffer and sorted with the flow cytometry method. The sorted cells were centrifuged, the obtained cell pellet was resuspended with Matrigel, and then cultured according to the “human liver organoid culture” protocol. 14 days after culturing, organoids larger than 100 μm were scored. Cells were cultured in Matrigel in 48-well (non-stick) cell culture plates as 3000-10000 cell/well. Once the Matrigel had solidified, EM (expansion medium/multiplication and maintenance medium) culture medium was added. EM culture medium consist of 1% N2 and 1% B27 without retinoic acid and 1.25 mM N-acetylcysteine, 10 nM gastrin and 50 ng/ml EGF, 10% RSPO1 conditioned cell culture medium (home-made), 100 ng/ml FGF 10, 25 ng/ml HGF, 10 mM Nicotinamide, 5 uM A8301 and Advanced DMEM/F12 with added 10 uM FSK. 25 ng/ml Noggin and 30% Wnt CM and 10 uM Y27632 were added to the culture medium during the first 3 days. The medium then continued with a culture medium which did not contain Noggin, Wnt and Y27632. Organoids were removed from Matrigel 10-14 days later, and they were mechanically split into pieces and transferred into a fresh matrix. Passaging was conducted every 7-10 days for at least 12 months at a ratio of 1:4-1:8. EpCAM (+) cells, which were defined for the first time by Hans Clevers and his team for the formation of adult human liver organoids and which grow in expansion medium (EM), started forming organoids from approximately day 3, and organoids with diameters larger than 100 μm were scored at the end of day 11. As a result of assays run with EpCAM (−) cells, it was seen that these cells do not have the ability to form organoids. Liver organoids were kept in cell culture medium containing 25 ng/ml BMP7 for 7-10 days in culture conditions explained above. Then the cultures were passaged and maintained 2-4 days more in the same medium. Then the medium was changed with DM (Differentiation medium), which consists of 1% N2 and 1% B27 without retinoic acid, 10 nM gastrin and 50 ng/ml EGF, 100 ng/ml FGF 10, 25 ng/ml HGF, 500 nM A8301, 10 uM DAPT, 25 ng/ml BMP7 and 30 uM Dexamethasone added developed cell culture medium DMEM/F12. This procedure was done every 2-3 days for a period of 10-14 days.

As a result, it was possible to enable the formation of more EpCAM (+) endoderm cells with this invention by means of modifying the endoderm differentiation medium. In addition to this, sorting of these EpCAM (+) cells provided a purer population, and thus, a very fast and effective organoid formation method. Moreover, liver organoids obtained through this method remained in culture longer than 48 passages and do not lose their liver functions even in later cultures.

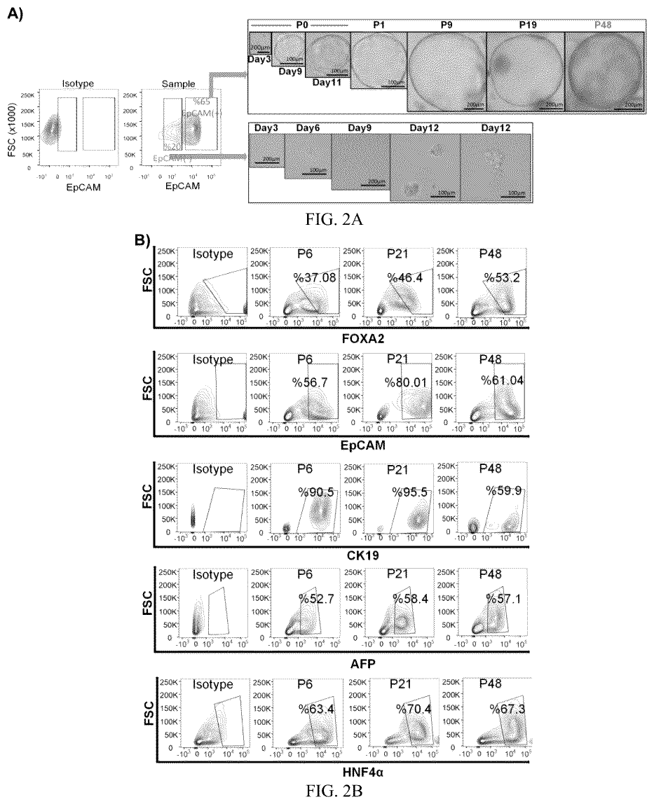

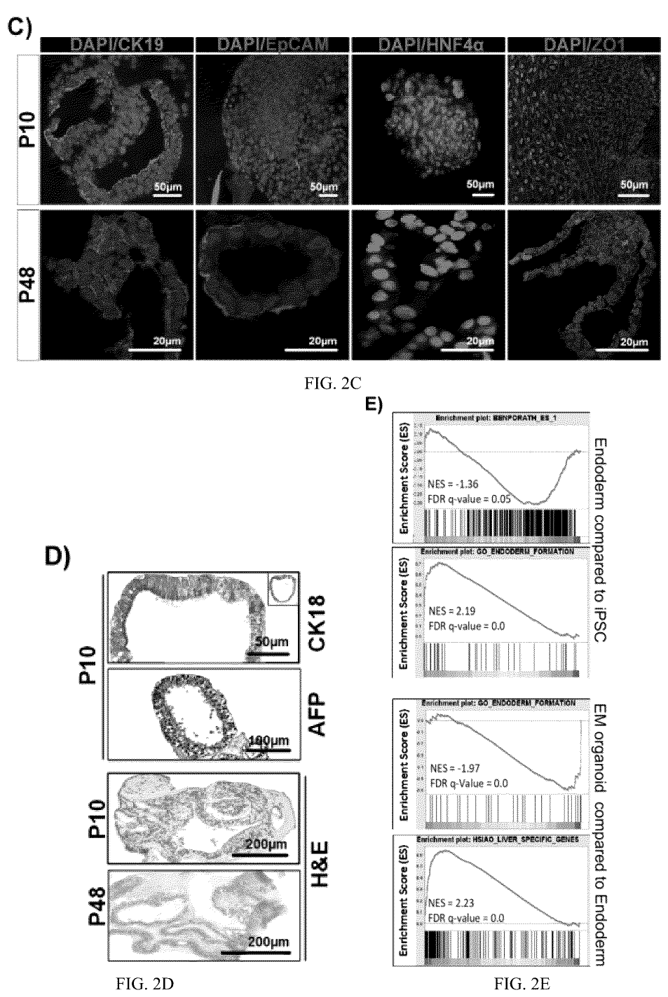

In a period as short as 1 week, EpCAM+endodermal cells were able to form three-dimensional empty structures resembling liver organoids derived from adult stem cells, whereas EpCAM− cells were deprived of this ability. When culturing EpCAM+derived organoids, structures with a round-shaped organoid morphology which had an approximate diameter of 100 nm and different sides were observed (FIG. 2A). Organoid cultures, in particular, were passaged every 7-10 days with a ratio of 1:5, and it was possible to culture them at late passages without a loss in phenotypical characteristics and differentiation capacity for 12 months. In order to identify the stability of EpCAM+endoderm cell-derived organoids cultured in EM medium conditions, different markers (AFP+, HNF4a+, FOXA2+, EpCAM+ and CK19+) were analysed at different time points (p6, 21 and 48) by flow cytometry. It was identified that the percentages of the subpopulations expressing EpCAM and FOXA2 (endoderm markers), a-fetoprotein (AFP) (fetal liver marker) and hepatocyte nuclear factor 4a (HNF4a) (hepatic marker) does not change considerably in different passages including young and old organoids. It was identified that the CK19 (hepatoblast/cholangiocyte marker) expression decreases in old organoids in EM conditions (FIG. 2B). These findings support the fact that in the long run, EM preserves the stable status of progenitor cells in organoid culture. In terms of the structural organisation, organoids at an early passage, CK19+ cells framing firmly self-organised and ductal-like structures, demonstrate that cells with hepatoblast and/or bile duct progenitor characteristics remain in these structures. For late passage organoids, it was identified that CK19+ cells rather spread in organoid- and ductal-specific locations equally. In addition, the continuing presence of EpCAM+ cells in organoids shows the permanence of liver precursor cells at early and late passages. HNF4a staining, in particular, indicated that organoids incline towards the hepatic strain of early and late passages, respectively. Furthermore, both early and late passage organoids have cubic/multifaceted epithelial cells which can express ZO-1 in a similar model, and thanks to tight junctions, they prove the existence of cell-cell interactions (FIG. 2C). Further immunohistochemical analysis of organoids showed that CK18+ and AFP+ cells form fake epithelial structures as seen in epithelial development (FIG. 2D). Haematoxylin/eosin staining demonstrates that typical structural organisation including fake (pseudostratified) epithelial and ductal-like structures does not change (at passage 48) even in long-term organoids (FIG. 2D). In order to understand the global differentiation of eHEPOs, RNA sequencing of IPSCs cultured in EM conditions, endodermal progenitors and organoids was conducted, as well as an independent characterisation of their identities on whole transcriptome level. Gene set enrichment analysis (GSEA) showed that the gene sets of gastrulation, endoderm formation and endoderm development are considerably richer than IPSCs in terms of endoderm induction. Pluripotency related genes, in contrast, were downregulated at the same stage (FIG. 2E). Following EM induction, endoderm-specific genes were downregulated and liver-specific gene sets were induced (FIG. 2E). When considered together, this data demonstrates that this protocol simulates the gradual development process of hepatic differentiation.

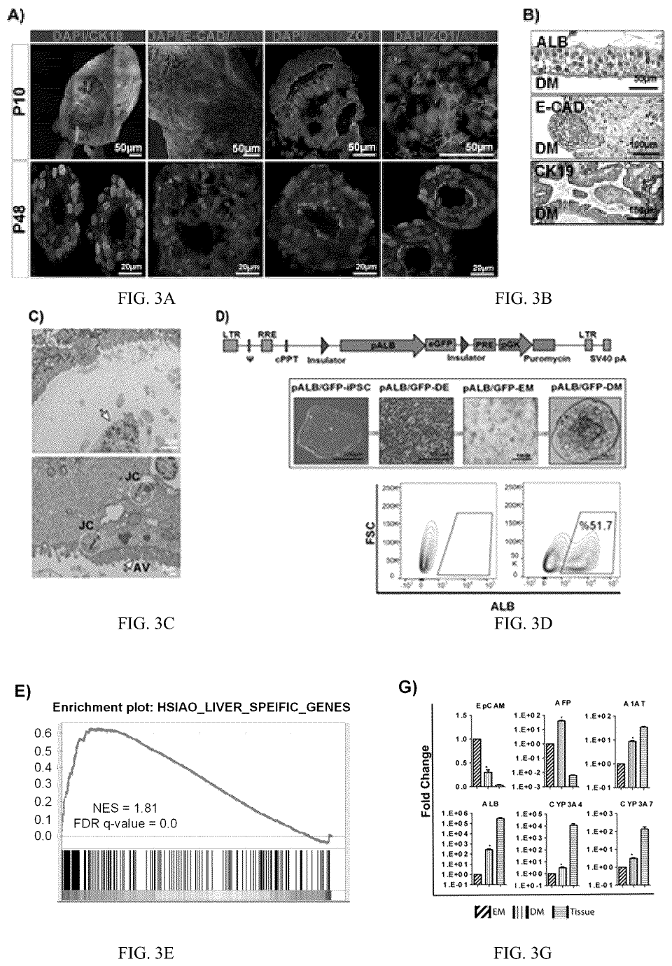

In order to advance the maturation of the obtained hepatic cells, organoids were cultured in differentiation medium (DM) for 10-14 days, and the expression of liver-specific genes and structural organisation were analysed, the latter through immunostaining. At this point, immunostaining was conducted for CK18, ZO-1, E-CAD, CK19, ALB and A1AT in DM conditions in both early passage (p10) and late passage (p48) organoids. All differentiated organoids consist of ALB+ and CK18+ hepatocytes which similarly have a typical polar structure, and the ZO-1 expression shows the presence of tight junctions separating apical and basolateral domains. Moreover, the E-CAD staining pattern shows the liver epithelium. ALB and A1AT staining provides evidence for hepatocyte maturation expressed even at late passages. IN the meantime, the presence of CK19+ cells particularly around lumen-like structures demonstrates the presence of cholangiocyte-like and/or progenitor cell population in differentiated organoids (FIG. 3A). Further immunohistochemical analyses revealed that organoids contain both ALB+ and CK19+ cells, which indicates mature hepatocytes with ductal-like structures and cholangiocytes, respectively. In addition, E-CAD+ cells represent polygonal epithelioid structures reflecting a hepatocyte-like phenotype (FIG. 3B). The ultrastructural analysis of organoids demonstrated the presence of a layer of live cells with an apical and basolateral polarity, as well as a lumen space containing the residue of apoptotic and multivesicular objects. The complex of intercellular junctions is defined by the characteristic of epithelial cells surrounding the lumen (FIG. 3C).

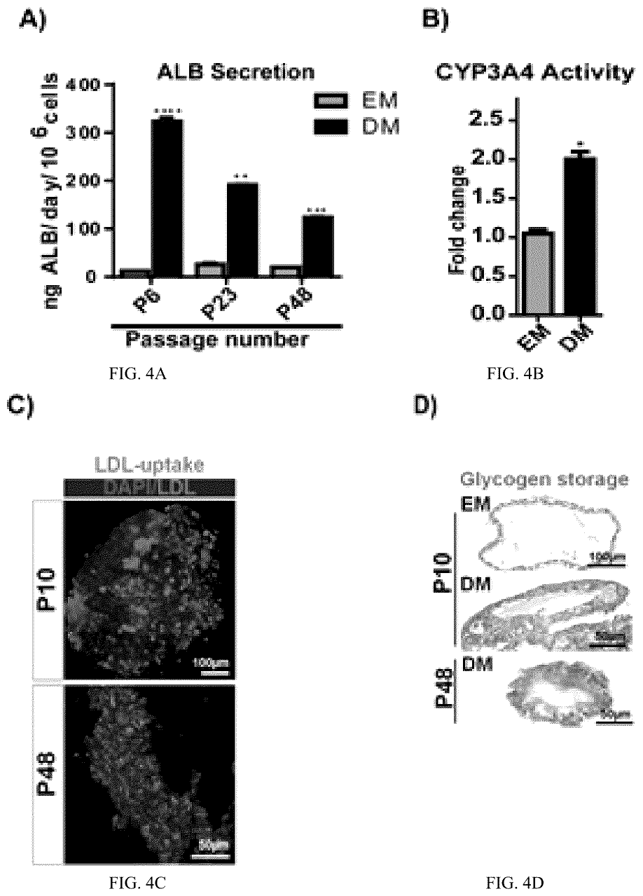

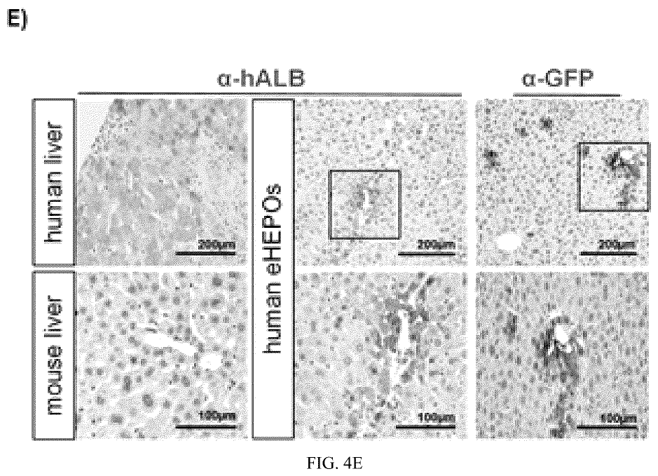

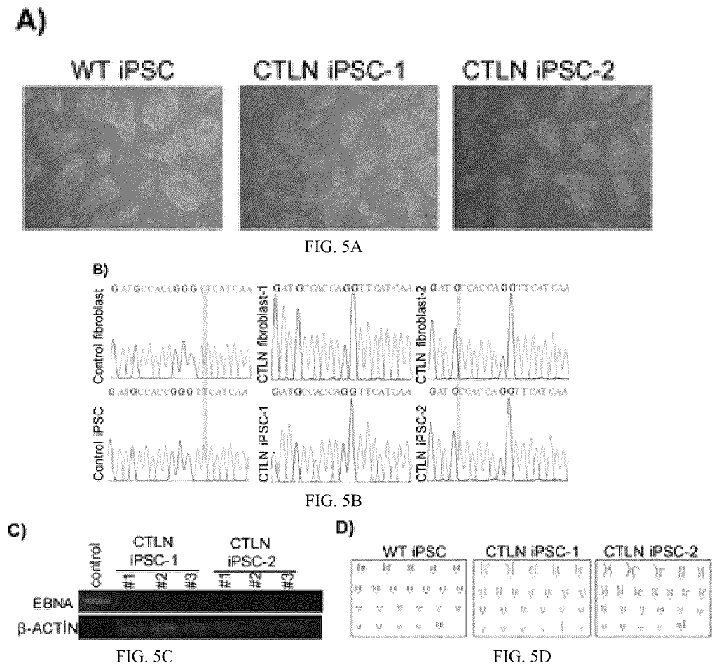

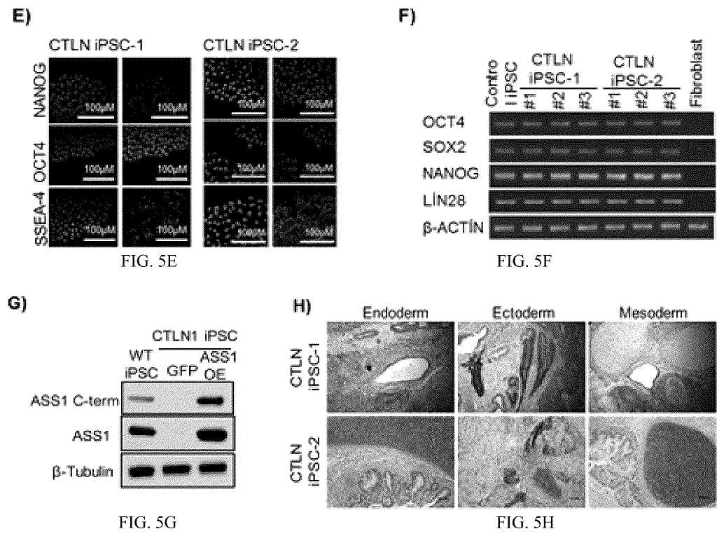

An albumin-GFP reporter system was developed to be able to characterise more the maturation of eHEPOs from IPSCs. An albumin enhancer/promoter conducting GFP expression was cloned to a lentiviral backbone between two flanking insulator elements, and following the integration into IPSCs, it was possible to observe the real-time differentiation of IPSCs into mature hepatocytes in organoids. Organoids were formed from reporter-carrying IPSCs, and it was identified that they become GFP positive after 5 days in DM culture conditions (FIG. 3D). A series of ALB+ cells in organoids formed from three independent differentiations starting from a single IPSC reporter line. There was not a major difference in the number of ALB+ cells (FIG. 3D). This data shows that organoid differentiation was successful. Global expression profiles were created after DM induction for a detailed assessment of the state of organoid differentiation. GSEA analysis demonstrated that liver-specific genes are highly upregulated in DM conditions (normalised enrichment score [NES] 1.81, false discovery rate [FDR] q value=0). This finding supports the hypothesis that liver-specific genes are much more upregulated in DM conditions when compared to EM conditions (FIG. 3E). The majority of key enzymes and receptors present in different aspects of liver function including glucose homeostasis (DCXR, IGFBP4, PGM1), lipid metabolism (RXRA, GHR, SOD1, APOC3, APOB, APOA1, LPIN1) and gluconeogenesis (PPP1R3B, GBE1) were induced on DM culture (FIG. 3F). qPCR validation of RNA sequencing data verified the upregulation of mature hepatocyte markers such as ALB, A1AT, CYP3A7 and CYP3A4 in organoids and the downregulation of endoderm stage marker EpCAM. However, gene expressions of organoids demonstrate a lower level when compared to adult human liver tissue (FIG. 3G). Mature eHEPOs in DM were shown to secrete a significant amount of albumin into the medium at different passages, which is known as an indicator of hepatocyte functionality. On the other hand, the level of albumin secreted in DM conditions decreased gradually based on the age of the organoid in the culture (FIG. 4A). Following differentiation, organoids also gained mature hepatocyte functions such as CYP3A4 activity, low-density lipoprotein (LDL) uptake and glycogen storage (FIGS. 4B-4D). Similar to organoids at early passages, eHEPOs at a late passage (p48) continued to display liver functions like LDL uptake and glycogen storage (FIGS. 4C and 4D). Cell injection trials were conducted to test the characteristics of differentiated organoids in in-vivo conditions, and firstly, a healthy IPSC cell carrying a GFP vector was formed. Then organoids were obtained from this IPSCs. Prior to cell injection into mice liver, the drug dimethylnitrosamine (DMN) was given to immunosuppressed NSG mice for 14 days to cause acute liver damage. Finally, 2 million eHEPO cells were intraspenically injected into mice with damaged livers, and thus, separate immunostaining of GFP and human-specific albumin antibodies showed the engraftment of human cells 32 days after transplantation (FIG. 4E). Human ALB+ cells were identified to be settled around interlobular veins and along the parenchyma. These results show that mature, functional hepatocytes obtained from eHEPO culture have the ability to engraft in mice liver. Research was conducted to see if it would be possible to use the eHEPO system for disease modelling later on. For this purpose, IPSC lines were formed from two patients who had new-born hyperammonemia and were clinically diagnosed with classic type 1 citrullinemia (CTLN1). CTLN1 is an autosomal recessive urea cycle disorder caused by the defects in the argininosuccinate synthase (ASS) enzyme due to mutations in the ASS1 gene. Patient-specific IPSCs were expanded feeder-free and showed typical pluripotent morphology (FIG. 5A). PCR amplification and sequencing of all coding exons of ASS1 showed that in exon 15, both fibroblasts and IPSCs of patients harbour homozygous G390R mutations, one of the most common mutations in classic citrullinemia. Where the nucleotide sequence of ASS1 gene in control fibroblasts is shown in SEQ ID NO: 1, the nucleotide sequence of ASS1 gene in CTLN fibroblasts-1 is shown in SEQ ID NO: 2, and the nucleotide sequence of ASS1 gene in CTLN fibroblasts-2 is shown in SEQ ID NO: 3; where the nucleotide sequence of ASS1 gene in control IPSC is shown in SEQ ID NO: 4, the nucleotide sequence of ASS1 gene in CTLN IPSC-1 is shown in SEQ ID NO: 5, and the nucleotide sequence of ASS1 gene in CTLN IPSC-2 is shown in SEQ ID NO: 6 (FIG. 5B). The generated IPSC lines lacked episomal vector sequences, as shown by genomic DNA PCR (FIG. 5C). An IPSC clone taken from each patient was further analysed with a chromosomal G band, and the fact that it has a normal karyotype was confirmed (FIG. 5D). CTLN IPSCs were positive for pluripotency markers OCT4, NANOG and SSEA-4 (FIG. 5E). RT-PCR analyses showed a high expression of OCT4, SOX2, NANOG and LIN28 mRNA in IPSCs derived from patients, however this was not seen in original dermal fibroblasts (FIG. 5F). As expected, ASS1 protein expression was identified in IPSCs derived from healthy donors, but not in patient-specific IPSCs (FIG. 5G). Finally, both CTLN-IPSC lines formed well differentiated teratomas containing cells derived from all three germ layers (FIG. 5H). When considered together, this data confirms the pluripotency of citrullinemia patient-based IPSCs.

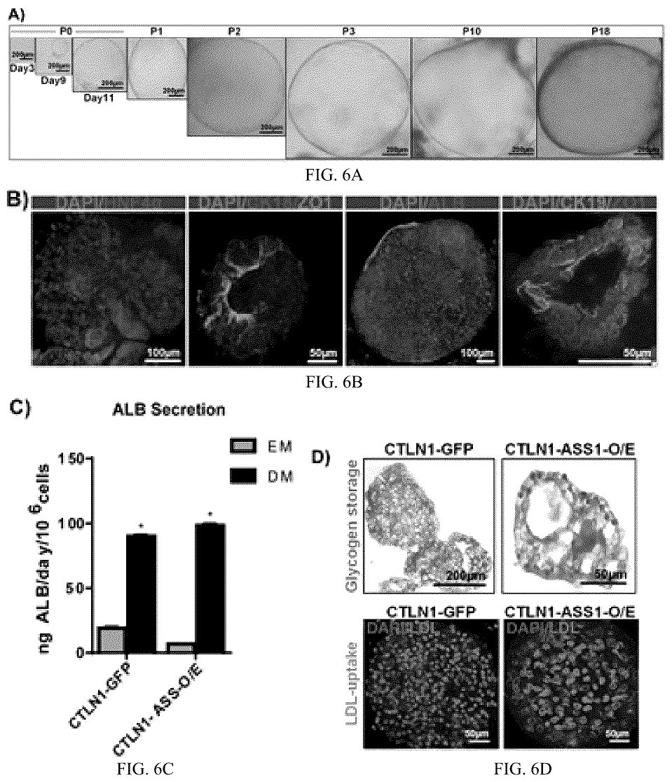

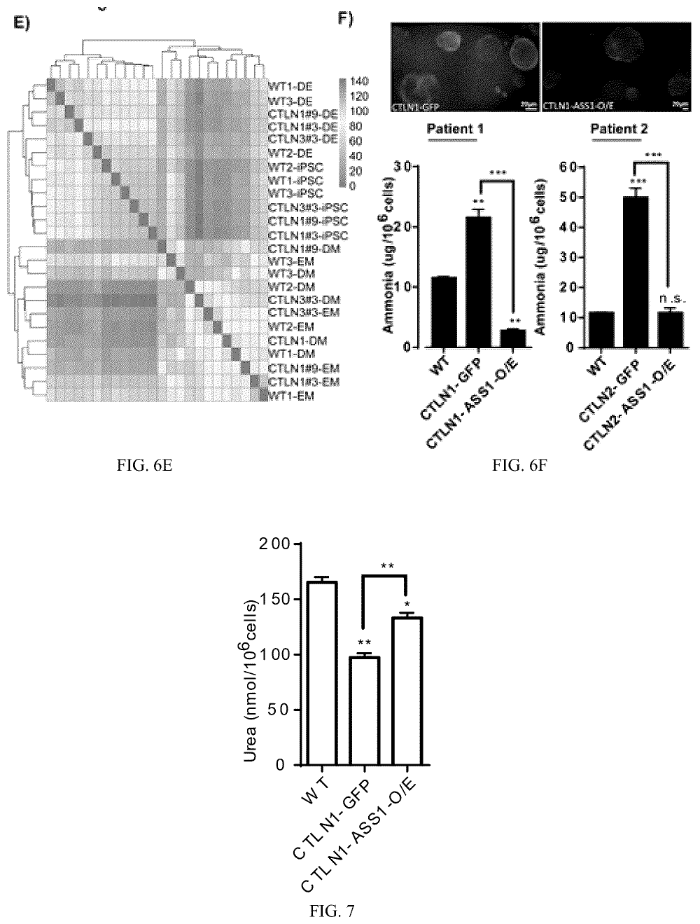

Following the protocol explained above, CTLN organoids that can last longer than 6 months in culture were successfully formed (FIG. 6A). Comparison of CTLN and wild-type organoids in terms of internal structural organisation revealed that both contain vectors. IPSCs derived from patients were transduced with an empty GFP vector to produce CTLN-GFP organoids as a control. Immunofluorescence staining of HNF4a, ZO-1, ALB, CK18 and CK19 for the phenotypical characterisation of eHEPO clones demonstrated that there is a similar model/structure between organoids derived from healthy donors and CTLN organoids (FIG. 6B). Liver functions defined for wild-type organoids (from healthy donors) were utilised in order to understand the hepatic maturation activity of the CTLN-GFP and CTLN-AS S1-O/E organoids. DM-mediated maturation caused a significant increase in albumin secretion for both eHEPO clones, which enabled a comparison of albumin levels (FIG. 6C). Furthermore, CTLN-GFP and CTLN-ASS1-O/E eHEPOs had LDL uptake and glycogen storage capacity in DM conditions (FIG. 6D). RNA sequencing of patient-derived cultures and k-means clustering of the Pearson correlation of whole transcriptomes demonstrated that organoids differ from less differentiated cell types in a clear way (FIG. 6E). Even though IPSCs and endodermal cells are similar in terms of molecular identity, they are still clustered separately, which shows that cell types can be appropriately distinguished (FIG. 6E). Most importantly, it was almost impossible to distinguish cells derived from patients from their healthy counterparts. DM culture of healthy and CTLN patient-derived organoids are almost identical (two genes that are FDR<0.01), which shows the CTLN disease-specific mutation does not affect the differentiation capacity of the patient-derived cells. ASS1 mutation causes ammonia accumulation in patients and decrease ureagenesis. Later, these phenotypes were studied in patient-derived eHEPOs, and whether or not the re-expression of wild-type ASS1 could save the disease-specific phenotypes in the hepatic organoid model was questioned. When compared to CTLN patient organoids, organoids derived from healthy donors displayed considerably less ammonia, whereas the re-expression of wild-type ASS1 in CTLN organoids rectified this flaw (FIG. 6F). Urea levels of the medium were measured accordingly, and it was seen that in contrast to healthy organoids, CTLN1-GFP organoids have a lower urea production capacity, and that the overexpression of ASS1 partially saves this phenotype (FIG. 7). When considered together, this data shows that hepatic organoids reflect the disease phenotype related to the urea cycle, and that the restoration of gene functions is possible in the eHEPO model.

1. A production method of a three-dimensional (3D) hepatic organoid obtained from induced pluripotent stem cells (IPSCs) comprising the steps of:
differentiating the IPSCs into a definitive endoderm in a medium containing Activin A, Wnt3a, and R-spo1 factors,
adding 5 ng/ml R-spo 1 during IPSC differentiation to increase an amount of EpCAM+endoderm progenitor cells,
cell sorting of the EpCAM+endoderm progenitor cells through a fluorescence-activated cell sorting (FACS) method,
culturing a sorted EpCAM+endoderm progenitor cells in a 3D Matrigel medium of 37° C., 5% CO2, 95% humidity and 7.2-7.5 pH,
once the 3D Matrigel medium solidifies, a cell culture medium of 1.25 mM N-acetylcysteine with 1% N2 and 1% B27 without retinoic acid, 10 nM gastrin and 50 ng/ml EGF, 10% RSPO1 is added to the 3D Matrigel medium, 100 ng/ml FGF 10, 25 ng/ml HGF, 10 mM Nicotinamide, 5 uM A8301, DMEM/F12 with added 10 uM FSK culture medium is added to the 3D Matrigel medium; during the first 3 days of this step, 25 ng/ml Noggin and 30% Wnt CM and 10 uM Y27632 is added to the 3D Matrigel medium,
for a functional hepatocyte differentiation of organoids, the 3D Matrigel medium needs to be changed with 1% N2 and 1% B27 without retinoic acid, 10 nM gastrin and 50 ng/ml EGF, 100 ng/ml FGF 10, 25 ng/ml HGF, 500 nM A8301, 10 uM DAPT, 25 ng/ml BMP7 and 30 uM Dexamethasone added a developed cell culture medium DMEM/F12 containing a differentiation medium.

2. A 3D hepatic organoid obtained according to claim 1.

3. The method according to claim 1, wherein the 3D hepatic organoid is obtained through the IPSC differentiation in laboratory culture medium.

4. The 3D hepatic organoid according to claim 2, configured for use in research towards a gene therapy planning and a discovery of drugs for a treatment of all liver metabolic diseases.

5. The 3D hepatic organoid according to claim 2, configured for use in a mimic Citrullinemia phenotype in vitro.

6. The 3D hepatic organoid according to claim 2, configured for use as a platform for a drug hepatotoxicity assay of drug candidates.

7. The 3D hepatic organoid according to claim 2, configured for use as a live hepatic function monitoring system to follow any harmful effect on human liver.


SEQUENCE LISTING (6 sequences)

SEQ ID NO: 1 - DNA, 20 bp, Artificial Sequence
  The sequence is synthetized.
gatgccaccgggttcatcaa

SEQ ID NO: 2 - DNA, 20 bp, Artificial Sequence
  The sequence is synthetized.
gatgccaccaggttcatcaa

SEQ ID NO: 3 - DNA, 20 bp, Artificial Sequence
  The sequence is synthetized.
gatgccaccaggttcatcaa

SEQ ID NO: 4 - DNA, 20 bp, Artificial Sequence
  The sequence is synthetized.
gatgccaccgggttcatcaa

SEQ ID NO: 5 - DNA, 20 bp, Artificial Sequence
  The sequence is synthetized.
gatgccaccaggttcatcaa

SEQ ID NO: 6 - DNA, 20 bp, Artificial Sequence
  The sequence is synthetized.
gatgccaccaggttcatcaa